(19) United States
(12) Patent Application Publication
(10) Pub. No.: US 2006/0271069 A1
(43) Pub. Date: Nov. 30, 2006

(54) Tick remover

(76) Inventor: Jorgen Glaesel, Hellerup (DK)

Correspondence Address: BIRCH STEWART KOLASCH & BIRCH, PO BOX 747, FALLS CHURCH, VA, 22040-0747, US

(21) Appl. No.: 10/539,255

(22) PCT Filed: Dec. 18, 2003
(86) PCT No.: PCT/DK03/00905

§ 371 Date: May 1, 2006

(30) Foreign Application Priority Data
Dec. 18, 2002 (DK) PA 2002 01931

(51) Int. Cl. A61B 17/50

(52) U.S. Cl. 606/131

(57) ABSTRACT

A tool (1) for removing ticks and the like parasites from the skin, said tool having an engagement part (2) with a bottom face (12) and a top face (14). A V-shaped groove (4) is provided that has side faces (11) between the bottom face and the top face at an edge (5) of the engagement part (2). The side faces (11) converge towards each other from an outer opening at the edge (5) to an inner assembly point (6), wherein the V-shaped groove (4) is wider at the top face (14) of the engagement part (2) that at the bottom side (12). An essential part of each side face (11) of the V-shaped groove (4) between the bottom face (12) and the top face (14) of the engagement part (2) is constituted of a concave engagement face (13).

The invention relates to a device for removing wood ticks and the like parasites from the skin, said device having an engagement part with an bottom face and a top face, a V-shaped groove being provided that has lateral faces between the bottom face and the top face at an edge of the engagement part, said side faces converging towards each other from an outer opening at the edge to an internal assembly point, said V-shaped grove being wider at the top face of the engagement part than at the bottom face.

To an increasing extent, wood ticks (and other like parasites) that carry pathogenic bacteria, such as borelia bacteria, are identified as the source of infection, and means by which wood ticks can be removed efficiently from the skin and without the pathogenic bacteria being transferred to the host are objects of increasing interest. Wood ticks thrive optimally in humid areas and during warm seasons and are found in particular in the shady vegetation of deciduous woodland, and humans as well as animals, eg dogs and deer, may be hosting ticks. Most recent research has shown that transfer of the pathogenic bacteria occurs if the tick transfers bacteria-infected secretion to the host. This may happen eg if it is attempted to remove it in an inappropriate manner and manipulates it for a protracted period of time, as this may cause the tick to disgorge or vomit. If still sitting on the host, such action will lead to transmission of the pathogenic bacteria to the host. It is therefore important that the tick is removed quickly and efficiently and the present invention provides a device for this.

U.S. Pat. No. 5,876,409 teaches a device as mentioned above in the form of a hook-shaped device with an engagement part in which a V-shaped groove is provided that has inclined engagement faces that extend from the bottom face of the engagement part to its top face. When a wood tick or other parasite is to be removed from the skin by means of this tool, the engagement part is displaced towards the wood tick thereby wedging it firmly into the V-shaped groove of the engagement part. Then the tool is rotated between one's thumb and index finger, which should make the wood tick let go after the second or third revolution of the tool. However, this tool is associated with the drawback that the tick is being manipulated for some seconds—on the one hand when the engagement part is to be caused to engage with the tick and, on the other, when the tool is rotated a number of revolutions. This means that there is a certain risk of the tick vomiting and thereby transmits pathogenic bacteria to the host.

U.S. Pat. No. 5,447,511 and U.S. Pat. No. 5,607,434 both teach comparatively flat tools for removing ticks. The tools are configured with a slightly curved engagement part having a V-shaped groove for receiving a tick. In use the tool is arranged on the skin and displaced until the head of the tick is wedged firmly between the side faces of the V-shaped groove. The tool is displaced further while simultaneously lifted, whereby it should be possible to readily remove the tick from the skin. The V-shaped groove in these tools is configured with side faces that are perpendicular to the bottom face of the engagement part. This means that the head and optionally the body of the tick is wedged firmly between the two side faces and the removal as such of the tick does not occur until the user subsequently displaces and lifts the tool. This manipulation of the tick prior to the concluding removal involves a risk that the tick has time to transmit pathogenic bacteria to the host before it is completely removed.

In order to avoid that the tick has time to transmit pathogenic bacteria it is thus crucial that it is removed in a quick and efficient manner without initial manipulation, which is precisely what makes the tick vomit and hence transmit the pathogenic bacteria.

It is therefore the object of the invention to provide a tool by which it is possible to remove wood ticks (and other like parasites) in a simple and efficient manner, while minimising the risk of transmission of pathogenic bacteria to the host.

This is accomplished by configuring the tool described above in such a manner that a considerable part of each side face of the V-shaped groove between the bottom face and the top face of the engagement part is constituted by a concave engagement face.

Configuration of the tool in this manner enables the formation of a hollow in the V-shaped groove of the engagement part with space for the head and body of the tick, thereby in most cases preventing these parts from being affected until the tick has been removed from the skin. This means that the risk of the wood tick having the time to transmit pathogenic bacteria to the host is more or less eliminated.

According to a particularly advantageous embodiment a lower part of the concave engagement face is essentially in parallel with the bottom face. Hereby it is accomplished that much space is generated transversally immediately above the bottom face, whereby influence on the head and body of the wood tick is avoided.

The concave shape of the engagement face may be formed of a continuously curved line segment that may have the shape of a drawing of a circle, or it may be formed of a plurality of straight lines. Irrespective of how the concave shape is generated, a space is formed between the side faces for the head and/or the body of the tick.

According to a preferred embodiment, each side face comprises a lowermost part that extends essentially perpendicularly upwards from the underside and is connected to that part of the side face that has a concave shape. By configuring the V-shaped groove in this manner the requisite rigidity and strength of the side edges of the V-shaped groove is obtained, thereby ensuring that they do not unintentionally flex outwards. It is further accomplished that the risk is reduced of the lowermost edge of the side faces cutting the skin when the tool is used for removing a tick or the like.

According to a preferred embodiment the lowermost part of the side face, seen from the edge to the assembly point, is, at least along an expanse, increasing in height.

Hereby a tool is accomplished by which, by one single sliding movement of the tool, the tick is lifted, while simultaneously it is wedged firmly between the lowermost parts of the side faces of the V-shaped groove. By lifting the wood tick the risk of the tick transmitting pathogenic bacteria is therefore further minimised.

Preferably the lowermost part of the side face has a constant low height a distance from the edge into the V-shaped groove. Hereby it is ensured that even small ticks are not wedged firmly between the side faces, but are rather seized from below by the engagement faces and lifted off the skin.

At the bottom of the V-shaped groove a cutter blade may advantageously be provided in level with the bottom face, whereby a tick that cannot be removed by the tool is at least cut off by a clean cut.

According to preferred embodiments the holder part of the tool according to the invention is provided with means for ensuring an ergonomically correct grip, which means may comprise an upwardly protruding transversal beam for supporting a thumb, an indentation for supporting a thumb, or a ribbed area for supporting a thumb, and/or at least one recess at the edge for supporting at least one finger.

According to the preferred embodiment the tool is plate-shaped, whereby, on the one hand, it is easy to store and, on the other, it is easy to impart thereto a desired flexibility for obtaining adequate and close contact with the skin in use.

In order to impart further flexibility to the tool, it may be an option to provide at least one recess in an area between the engagement part and the holder part.

According to a particular embodiment, the largest outer dimension of the tool corresponds essentially to the dimensions of a credit card. Hereby the tool is readily kept along with credit cards and other cards of same standard dimension.

The invention will now be explained in further detail with reference to the drawing, wherein

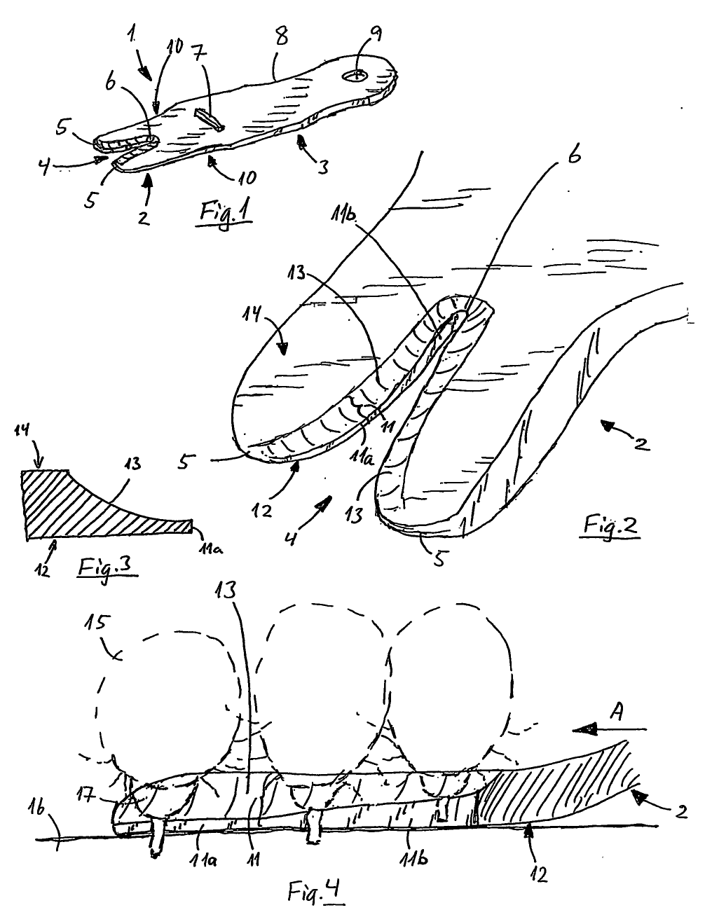

FIG. 1 shows a tool according to the invention in a first embodiment;

FIG. 2 is an enlarged view of the engagement part of the tool shown in FIG. 1;

FIG. 3 is a cross sectional view of the side face of the V-shaped part of the engagement part shown in FIG. 2;

FIG. 4 is a cross-sectional view through the engagement part shown in FIG. 2 during removal of a tick;

FIG. 5 shows how the tool shown in FIG. 1 is used to remove a tick;

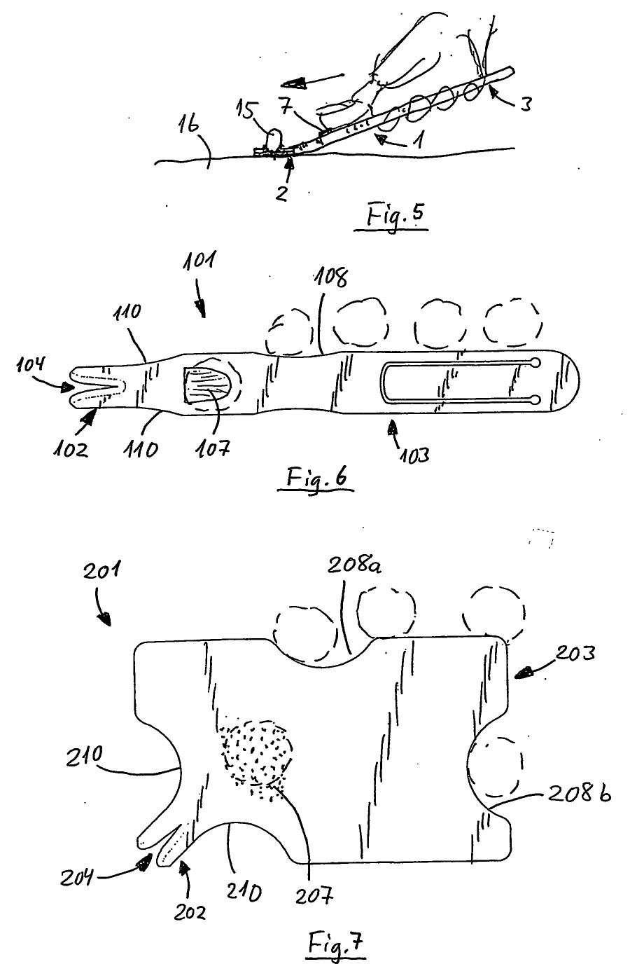

FIG. 6 shows a tool according to the invention in a second embodiment;

FIG. 7 shows a tool according to the invention in a third embodiment;

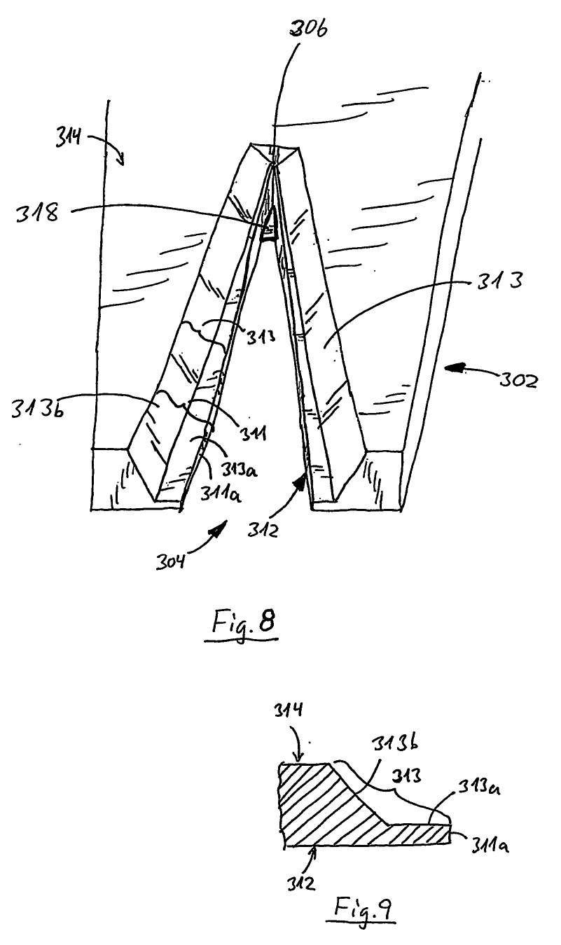

FIG. 8 shows an alternative embodiment of the engagement part of a tool according to the invention; and

FIG. 9 is a cross sectional view of the side face of the V-shaped groove in the engagement part shown in FIG. 8.

FIG. 1 shows a tool 1 for removing ticks and the like parasites according to the invention in a first embodiment. The tool 1 comprises an engagement part 2 and a holder part 3; the engagement part 2 being configured with means for removing a tick from the skin of a host, and the holder part 3 being configured with means for accomplishing optimal seizing of the tool 1 during use.

The engagement part 2 is provided with a V-shaped groove 4 that extends from the edge 5 of the engagement part 2 to an assembly point 6, and in the preferred embodiment it is configured as shown more detailed in FIGS. 2-4, which will be subject to more detailed description later in this description.

In the embodiment shown in FIG. 1, the holder part 3 comprises an upwardly protruding transverse beam 7 that serves as stop for the user's thumb and a recess 8, the edge of which serves as a stop for the user's index finger when the tool 1 is used for removing a tick. In the embodiment shown, the holder part 3 is further provided with a through-going aperture 9, thereby enabling the tool 1 to be carried in a chain or string that extends through the aperture 9, or it can be inserted into a key ring.

As will appear the engagement part 2 is narrower than the holder part 3, since recesses 10 are provided that make the engagement part 2 of the tool 1 more flexible than the holder part 3. This is of consequence to the functioning of the tool 1, as will appear from the below disclosures.

The tool 1 is preferably made of a comparatively hard, yet flexible plastics material; however, it may also be manufactured from eg metal.

FIG. 2 shows the engagement part 2 of the tool 1 shown in FIG. 1 in an enlarged scale. As will appear, the V-shaped groove 4 is configured from the edge 5 of the engagement part 2. The edge 5 is rounded to minimise the risk of damaging the skin when the tool 1 is used, but also to minimise the risk of harming or destroying the tick during use of the tool 1.

The V-shaped groove 4 is configured with opposing side faces 11 that extend from the bottom side 12 of the engagement part 2 to its top side 14. The lowermost part 11a of the side face 11 extends essentially perpendicularly upwards from the bottom face 12 of the engagement part 2. Each side face 11 further comprises an engagement face 13 that extends from the lowermost part 11a of the side face 11 to the top side 14 of the engagement part 2. As will appear the transition between the lowermost part 11a of the side face 11 and the engagement face 13 extends in the outer part of the V-shaped groove 4, essentially in parallel with the bottom face 12 of the engagement part 2, while—further into the V-shaped groove 4—it diverges from the bottom face 12. This means that the distance between the engagement face 13 and the bottom face 12 increases the closer the engagement face 13 is to the assembly point 6.

The V-shaped groove 4 may be configured with straight side faces seen from above; preferably, however, it is configured with curved side faces as shown in FIG. 2. The advantage of using curved side faces instead of straight side faces is that it is hereby possible to configure the engagement part 2 in such a manner that the most careful removal of ticks is ensured. Hereby the risk is reduced of the tick having the time to transmit pathogenic bacteria prior to being completely removed.

In accordance with the invention, the side face 11 is provided with a concavely configured engagement face 13, as will appear in a perspective view in FIG. 2, and yet more clearly from the sectional view shown in FIG. 3. When the engagement face 13 is made to be concave, a hollow is provided that provides ample space for receiving the head and/or body of a tick without it being squeezed between the opposing engagement faces 13 of the V-shaped groove 4. The concave shape is preferably configured like the drawing of a circle; yet other configurations, however, are possible, see the below teachings.

In the embodiment shown in FIG. 2 the innermost segment of the V-shaped groove 4 is provided with parallel side faces 11b and with a rounded bottom at the assembly point 6. This configuration ensures that small ticks that are not seized by the engagement faces 13 are not merely squeezed between the engagement part 2, but are rather conveyed into this slot and are subsequently pushed by or cut off by the bottom side 12 of the engagement part 2 at the bottom of the V-shaped groove 4.

In the following the use of the tool 1 will be described more closely, reference being made to FIG. 4 that shows a cross-section through the engagement part 2 during removal of a tick 15 from the skin 16 of a host; and to FIG. 5 that shows how the tool 1 is held during removal of a tick 15.

Firstly the position of the tick 15 on the skin is located, and as much hair or pelt as possible is pushed aside without the tick 15 being touched. Then the tool 1 is seized by the hand, the thumb being caused to abut on the upwardly protruding transverse beam 7 and the index finger being caused to abut on the recess 8. The engagement part 2 of the tool 1 now presses down towards the skin 16 with the opening of the V-shaped groove pointing towards the tick 15. Owing to the recesses 10, the tool 1 flexes in the transition between the engagement part 2 and the holder part 3, as shown in FIG. 5.

The engagement part 2 of the tool 1 is now displaced in a swift movement towards and past the tick 15, whereby it is removed—the transition between the engagement faces 13 and the lowermost parts 11a of the side faces 11 engaging with the head 17 of the tick 15 and lifting the tick 15 off the skin 16, while the tool is being displaced across the skin. This is shown in FIG. 4 that shows, by a fully drawn line, the initial position of the engagement part 2 in relation to the tick 15 in a situation where no contact has yet been established between the head 17 of the tick 15 and the transition between the engagement faces 13 and the lowermost parts 11a of the side faces 11. When the engagement part 2 is displaced in the direction of the arrow A across the skin 16, the transition between the engagement faces 13 and the lowermost parts 11a of the side faces 11 engage with the head 17 of the tick, and as the distance between the engagement face 13 and the underside of the engagement part 2 is increased towards the assembly point 6, the tick 15 will be lifted off the skin 16 as outlined by the dotted ticks.

In use the tool 1 is conveyed across the skin 16 in a swift, uninterrupted movement, and the elevation of the tick 15 therefore takes place within fractions of a second. Conversely to the known tools for removing ticks, use of the tool 1 having engagement faces 13 that move in a direction away from the bottom face 12, means that the tick 15 is lifted off the skin 16. This means that it is not manipulated in an adverse manner and thereby the risk of it transmitting pathogenic bacteria is minimised.

In order to ensure that the tool according to the invention can be used effectively, it is preferably provided with an ergonomically correctly configured holder part. It may be accomplished by configuring the holder part as shown in FIG. 1, where a transverse beam 7 and a recess 8 serve as stop for the user's thumb and index finger as described above.

An alternative embodiment of a tool according to the invention is shown in FIG. 6 in the form of a pen-shaped tool 101. This tool 101 is plate-shaped throughout its entire length like the tool 1 shown in FIG. 1 and it is further provided with a corresponding V-shaped groove 104. The holder part 103 is configured with an indentation 107 for the thumb and with a recess 108 for the index finger. The positioning of the fingers is outlined by dotted lines in FIG. 5. This embodiment, too, features recesses 110 in the transition between the engagement part 102 and the holder part 103 to increase the flexibility in this area.

According to a particular embodiment of a pen-shaped tool, the holder part has a generally round or oval cross-section, while the engagement part is preferably still plate-shaped. Such embodiment, however, is not shown.

An alternative embodiment of a tool according to the invention is shown in FIG. 7 that shows a plate-shaped tool 201 having an outer contour of the same size as a credit card. The tool 201 is preferably manufactured from plastics having the same thickness as a credit card, but it may also be manufactured from metal. The tool 201 comprises an engagement part 202 in the one corner, said engagement part 202 being provided with a V-shaped groove 204 configured in accordance with the invention.

This tool, too, is configured with recesses 210 that enable the engagement part 202 to be flexed relatively easily in relation to the holder part 203 of the tool 291. The holder part 203 is moreover configured with recess 208a for the index finger of the user and with recess 208b for the user's little finger. Finally a ribbed area 207 is provided for the thumb of the user. The user's fingers are outlined by dotted lines, and the provision of the recesses 208a, 208b and the ribbed area 208 enables the user to obtain a safe grip around the tool 201 during use.

Advantageously this embodiment can be provided with two engagement parts 202 arranged in two neighbouring corners since it can hereby be obtained that the tool can be used in the same manner with both one's right and one's left hand.

As will appear from the above and from FIGS. 1, 4, 5 and 6 it is of interest to provide the tool 1, 101, 201 with means that ensure an ergonomically safe grip around the tool during use. This is to be seen in the context of the tool 1, 101, 201 often being used in circumstances where the user has wet or slippery fingers. If the tool is configured with smooth outsides without particular recesses or other means that may act as stops for one or more fingers, there is a risk of the tool slipping in the user's hand and thus it does not remove the tick effectively.

FIG. 8 shows an alternative embodiment of the engagement part 302 for a tool according to the invention. In this embodiment the engagement part 302 is configured with engagement faces 313 that comprise a first part 313a which is in parallel with the bottom face 312 of the engagement part 302 and a second part 313b that extends as a plane face from the first part 313a to the top face 314 of the engagement part 302. By this configuration, the engagement face 313 becomes concave as will appear most clearly from FIG. 9, which shows a cross-sectional view of the engagement face 313 in the engagement part 302 shown in FIG. 8. By the concave configuration of the engagement faces 313, a space is formed between the engagement faces 313, whereby a tick can be removed without its head and/or body being influenced laterally as described above.

Also in this embodiment the transition between the engagement faces 313 and the lowermost part 311a of the side faces 311 diverge in a direction towards the assembly point 36 of the V-shaped groove 304 from the bottom side 312 of the engagement part 302. The functioning of the engagement part 302 corresponds in principle to the functioning of the engagement part 2 shown in FIGS. 2-4, ie the head of the tick is lifted upwards by the engagement faces 313 when the V-shaped groove 304 is displaced around the tick.

In the embodiment shown in FIG. 8, the engagement part 302 is, at the bottom of the V-shaped groove 304, provided with a cutter blade 318 that may consist of metal or other suitable material. The functioning of the cutter blade 318 is to cut off the head of the tick if, contrary to expectations, it is not accomplished to lift the tick off the skin. This may occur if the tick is extremely firmly embedded or if it is so small that the engagement faces 313 are unable to impart a sufficient lift to the tick.

If the tool for removing ticks is manufactured from an injection moulded plastics material, the cutter blade 318 may be configured as a integrally moulded knife's blade or razor blade, or the cutter blade 318 may be provided as an integral plastics part which is moulded with a very sharp front edge. Such cutter blade may of course also be provided at the bottom of the V-shaped groove in the embodiments shown in FIGS. 1-7.

The invention was described with reference to various embodiments of both the engagement part as such and the overall configuration of the tool. However, both of these elements can be varied beyond the disclosures of FIGS. 1-9. However, the tool is preferably configured as a relatively flat object as shown in FIGS. 1-9, whereby flexibility around the engagement part can easily be incorporated, whereby adequate and close contact with the skin during use is obtained. Moreover, the plate-shaped configuration means that the tool can easily be stored in various ways, eg in a pocket or along with the user's credit cards. However, nothing prevents the tool from being configured as a thicker unit, as long as care is being taken to ensure that the engagement faces are configured to be concave, thereby forming a hollow for receiving the head and/or body of the tick when the engagement part is displaced across it.

The tool is preferably manufactured by injection moulding of a plastics material that can be transparent or coloured. Alternatively the tool can be made of plastics or glass.

1. A tool (1; 101; 201) for removing ticks (15) and other like parasites from the skin (16), which tool has an engagement part (2; 102; 202; 302) with a bottom face (12; 312) and a top face (14; 314), wherein a V-shaped groove (4; 104; 204; 304) is provided that has side faces (11; 311) between the bottom face and the top face at an edge (5) of the engagement part (2; 102; 202; 302), which side edges (11; 311) converge towards each other from an outer opening at the edge (5) towards an internal assembly point (6; 306), said V-shaped groove (4; 104; 204; 304) being wider at the top face (14; 314) of the engagement part (2; 102; 202; 302) than it is at the underside (12; 312), characterised in that a considerable portion of each side face (11; 311) of the V-shaped groove (4; 104; 204; 304) between the underside (12; 312) and the top face (14; 314) of the engagement part (2; 102; 202; 302) is constituted by a concave engagement face (13; 313).

2. A tool according to claim 1, characterised in that a lowermost part of the concave engagement face (13; 313) is essentially in parallel with the bottom face (12; 312).

3. A tool according to claim 1, characterised in that the concave shape of the concave engagement face (13) is formed by a continuously curved line segment.

4. A tool according to claim 3, characterised in that the concave shape of the concave engagement face (13) is formed by a drawing of a circle.

5. A tool according to claim 1, characterised in that the concave shape of the concave engagement face (13) is formed by number of straight lines.

6. A tool according to claim 1, characterised in that each side face (11; 311) comprises a lowermost part (11 a, 11 b; 311 a) that extends essentially perpendicularly from the bottom face (12; 312) and is connected to the concave engagement face (13; 313).

7. A tool according to claim 6, characterised in that, at least for a distance, the lowermost part (11 a, 11 b; 311 a) of the side face (11; 311) is increasing in height, seen from the edge (5) to the assembly point (6; 306).

8. A tool according to claim 7, characterised in that the lowermost part (11 a, 11b; 311 a) of the side face (11; 311) has a constant low height a distance from the edge into the V-shaped groove (4; 104; 204; 304).

9. A tool according to claim 1, characterised in that an innermost part of the lowermost part (11b) of the side face (11) is provided with parallel side faces.

10. A tool according to claim 1, characterised in that, at the bottom of the V-shaped groove (304) a cutter blade (318) is provided in level with the bottom face (312).

11. A tool according to claim 1, characterised in that it comprises a holder part (3) provided with an upwardly protruding transverse beam (7) for supporting a thumb.

12. A tool according to claim 1, characterised in that it comprises a holder part (103) provided with an indentation (107) for supporting a thumb.

13. A tool according to claim 1, characterised in that it comprises a holder part (203) provided with a ribbed area (207) for supporting a thumb.

14. A tool according to claim 1, characterised in that it comprises at least one recess (8; 108; 208a; 208b) at the edge in support of at least one finger.

15. A tool according to claim 1, characterised in being plate-shaped.

16. A tool according to claim 15, characterised in that at least one recess (10; 110; 210) is provided in the area between the engagement part (2; 102; 202) and the holder part (3; 103; 203).

17. A tool according to claim 15, characterised in that the largest outer dimension of the tool corresponds essentially to the dimensions of a credit card.
